(19) United States
(12) Patent Application Publication
(10) Pub. No.: US 2006/0135568 A1
(43) Pub. Date: Jun. 22, 2006

(54) Methods of treating microbial infections in humans and animals

(76) Inventors: Craig A. Townsend, Baltimore, MD (US); James D. Dick, Baltimore, MD (US); Nicole M. Parrish, Ellicott City, MD (US); Minerva Amorette Hughes, Baltimore, MD (US)

Correspondence Address: COVINGTON & BURLING;ATTN: PATENT DOCKETING, 1201 PENNSYLVANIA AVENUE, N.W., WASHINGTON, DC, 20004-2401, US

(21) Appl. No.: 10/520,506

(22) PCT Filed: Jul. 9, 2003
(86) PCT No.: PCT/US03/21469

§ 371 Date: Nov. 1, 2005

Related U.S. Application Data
(60) Provisional application No. 60/394,573, filed on Jul. 9, 2002.

(51) Int. Cl. A61K 31/455; A61K 31/421; A61K 31/381; A61K 31/365; A61K 31/16; A61K 31/22

(52) U.S. Cl. 514/355; 514/374; 514/445; 514/471; 514/550; 514/614

(57) ABSTRACT

A method of treating a subject with a microbially-based infection, comprising the administration of a compound to the subject. The compound is able to decrease ATP levels in the microbe by at least 10% compared to controls after 24 hours in an in vitro test, without killing mammalian cells during the same time period. The decrease in ATP levels is measured by: (1) removing the cells from the testing location and putting them on ice; (2) harvesting the cells at 4 degrees C. by centrifugation and disrupting it with bead-beating in an ATP extraction buffer; (3) removing cellular debris by centrifugation at 4 degrees C., leaving an ATP-containing supernatant; (4) measuring the amount of ATP present in the supernatant by a bioluminescence assay at 4 degrees C.

### BACKGROUND OF THE INVENTION

Microbially-based infections remain a major public health issue in the United States and around the world. For example, tuberculosis remains a significant health problem in the U.S. and globally. Tuberculosis (TB) is the leading cause of death due to a single infectious agent in the world. It is believed that approximately 1.86 billion people or 32% of the world's population are infected with Mycobacterium tuberculosis (M. tb.) There are about 8 million new active cases of TB per year and approximately 2 million deaths. This translates into a mortality rate of 200 people every hour and 5000 people every day. Patients with HIV infection demonstrate a significantly increased susceptibility to M. tb. with an approximate 50-fold risk increase over patients without HIV (12, 45). Similarly, the rate of progression of latent TB to active disease following initial infection is greater that 40% compared to approximately 5% in HIV-uninfected individuals. With the continued expansion of HIV globally, particularly in Asia and the Indian sub-continent, the incidence and mortality of TB can only be expected to increase.

The increasing incidence in M. tb. strains resistant to one or more of the standard first-line agents intensifies the need for the identification of new, novel targets and drug development. MDR-TB (multi-drug resistant tuberculosis) difficult and expensive to treat, as well as being associated with significantly higher mortality rates than drug-susceptible TB. In the absence of effective prevention and therapeutic measures, MDR-TB will become an increasing and uncontrollable problem.

A significant need exists for improved tuberculosis drugs with reduced toxicity, activity against MDR-TB, alternate mechanisms of action, and activity against latent disease. Despite advances in the prevention and treatment of tuberculosis over the past five decades, significant obstacles remain before control of this disease can be anticipated. Current standard of care strategies are difficult to implement and maintain, particularly in low-income, non-industrialized countries, which lack the financial resources or infrastructure to support an effective or all-inclusive TB control program. The emergence of MDR-MB threatens to reverse much of the progress made to date in TB control.

Several promising drug classes are under development including long-acing rifamycins, fluoroquinolones, oxazolidinones, and nitroimidazoles. Nevertheless, given the success rate of new compounds coming to clinical use—approximately 0.5%—there is a need for discovery and identification of new unique mycobacterial targets for development. No new antimycobacterial drugs with novel mechanisms of action have been developed in the past thirty years.

### OBJECTS OF THE INVENTION

It is an object of this invention to provide a method of treating a microbially-based infection by administration of a compound which interferes with the central energy metabolism of the microbes.

A further object of the invention comprises administration of a compound which inhibits ATP synthesis in microbes and which interferes with cellular respiration of such microbes.

A further object of the invention comprises administration of a compound which will cause a decrease in ATP[M] levels of at least 10% relative to control.

A further object of the invention comprises a method of treating a subject with a microbially-based infection, comprising the administration of an effective amount of compound to a subject in need of treatment, wherein the compound produces overexpression of the b-subunit of ATP synthase.

A further object of this invention is to provide certain compounds which, when administered to persons or animals with a microbial infection, can treat the infection through the above-described mechanisms:

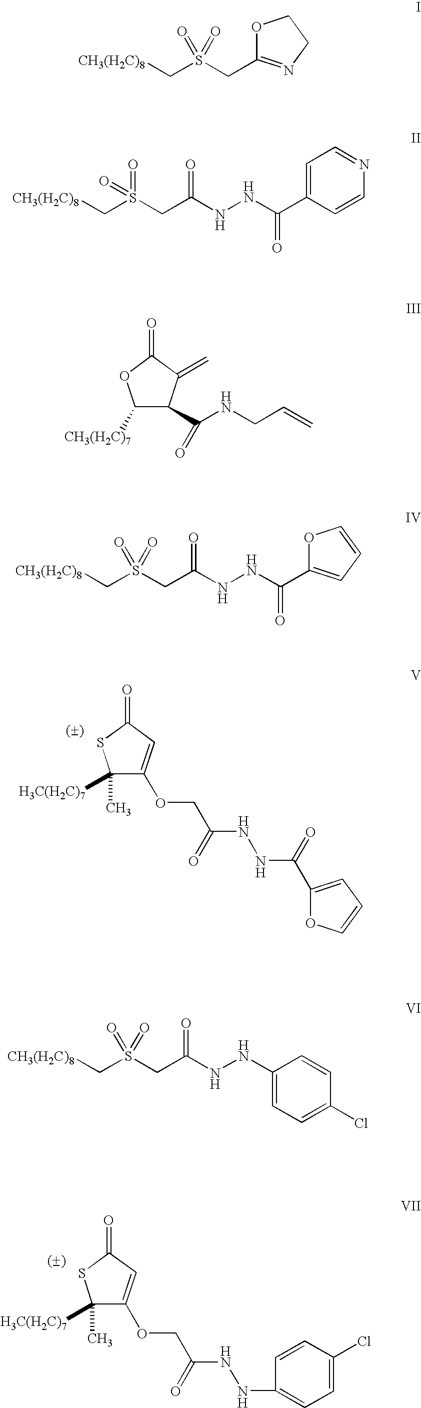

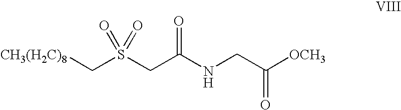

### BRIEF DESCRIPTION OF THE DRAWING

FIG. 1 shows the general structure and function of ATP synthase.

FIG. 2 shows two-dimensional protein gel electrophoresis profiles of control vs. OSA treated (100 μg/ml) BCG at 4 hours post treatment.

FIG. 3 Expression comparison of atpf, encoding the b-subunit of ATP synthase (Rv1306) and hsp, (Rv0251c) in BCG grown in the presence or absence of OSA.

FIG. 4 Time-course experiment measuring ATP level in BCG cultures treated with OSA or dicyclohexylcarbodiimide compared to untreated controls.

FIG. 5 ATP concentration/CPU in BCG following 5 minutes of exposure to OSA, known inhibitors of respiration and antimycobacterial agents.

FIG. 6 shows the potentiation of the inhibitory activity of OSA at low concentrations of ethanol (0.05%) against M. tuberculosis.

FIG. 7 shows a comparison of the effects of OSA (100 μg/ml), DCCD (100 μg/ml), and TTFA (100 μg/ml) on mycolic acid synthesis following 10 minutes exposure in early log phase cultures of BCG.

### DETAILED DESCRIPTION OF THE INVENTION

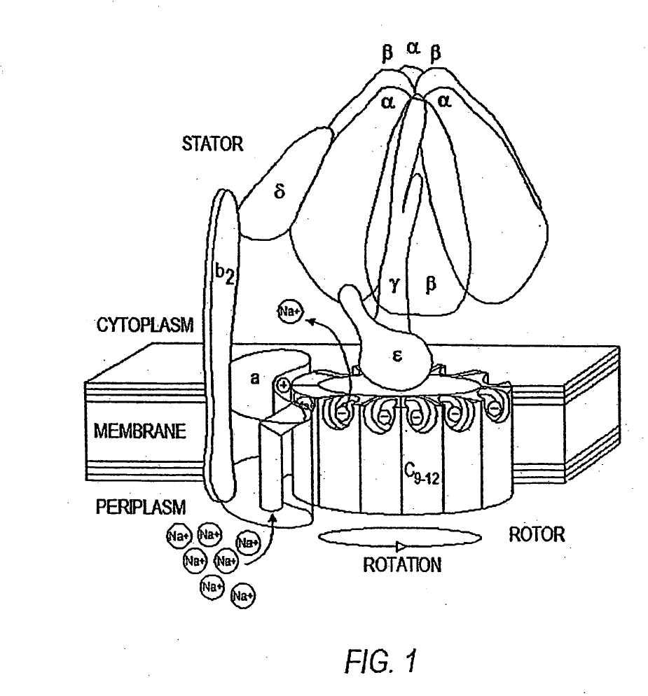

FIG. 1, which is derived from Dimroth, et al., “Operation of the F(0) motor of the ATP synthase,” (2000) 1458: 374-386 shows schematically the structure of F1F0 ATP synthase (ATPase). ATPase uses energy from the proton motive force to generate ATP. This enzyme complex consists of transmembrane (F0) and cytosolic sectors (F1). The movement of protons through the F0 component, is thought to be reversibly coupled to ATP synthesis or hydrolysis in catalytic sites on F1. In E. coli, F1 and F0 consist of the following subunits, α₃β₃γδε, and a₁b₂c₁₂, respectively. In general, homologous subunits are found in mitochondria and chloroplasts, although differences between prokaryotic and eukaryotic systems may exist ATP synthesis is driven by proton movement through F0. However, the complete structure and mechanism of coupling has not been fully established.

Cross-linking of the b-subunit with another F0 membrane component (subunit c) in E. coli resulted in uncoupling of ATP hydrolysis and ATP-driven proton pumping. Similarly, an uncoupling mutation has been found in E. coli affecting the b-subunit of F0 involving a single amino acid substitution, which abolished al, enzyme function. This phenotype suggests a functional role for the b-subunit in coupling of proton translocation to catalysis. Interaction of the b-subunit with components of F1 may be both dynamic and structural in nature. More recently, Struglics and coworkers have shown the b-subunit of mitochondria to be reversibly phosphorylated. The authors suggest that the physiological role of such phosphorylation may control the stability of the F0-F1 interaction and thereby regulate energy coupling in the F0-F1 motor. As such, the b-subunit of F0 would play both a structural and functional role in operation of ATPase. Inhibition of this particular complex could occur through direct interaction with the b-subunit or a membrane associated component of F0 resulting in a significant impairment of ATP generation.

Significant study of this possible mechanism has been undertaken with n-octanesulfonylacetamide (OSA) a compound of the □-sulfonylcarboxamides class, which has potent in vitro activity against pathogenic mycobacteria. OSA is disclosed in PCT Application No. PCT/US98/17830, which is incorporated herein by reference.

Identification of the enzymatic target of OSA in bacillus Calmette-Guérin (BCG) was attempted using 2-dimensional protein gel electrophoresis and subsequent sequencing of proteins overexpressed in the presence of the compound. OSA treatment in BCG resulted in overexpression of 2 relatively small proteins (≈18 kD): the b-subunit of ATP synthase (F1F0 ATPase) encoded by the atpF gene and a small heat shock protein, hsp (Rv0251c). RT-PCR revealed a marked increase in the level of hsp expression and to a lesser extent the b-subunit of ATP synthase, a pattern consistent with that observed on the 2D gels.

To evaluate whether these results might represent a generalized stress response to cell injury, 2-dimensional protein profiles were carried out in the presence of cerulenin, a potent antimycobacterial compound, and isoniazid, another potent anti-TB compound. Neither cerulenin, not isoniazid treatment resulted in overexpression of either protein in BCG, indicating that OSA works via a different mechanism than either cerulenin or isoniazid.

Additional information was obtained by comparing 2-dimensional protein profiles of OSA-treated M. smegmatis with BCG. Previously, it had been reported that M. smegmatis was intrinsically resistant to OSA at concentrations up to the limit of solubility (100 μg/ml). Homologs of both BCG proteins were sought by a BLAST search of the M. smegmatis genomic sequence available through The Institute for Genomic Research, Rockille, Md. (http//www.tigr.org) and found to be present in M. smegmatis. However, regions of dissimilarity between the two exist. The b-subunit looks fairly similar between these two mycobacterial species (63% identical, 75% similar), however, the hsp is less so (42% identical, 54% similar). The estimated molecular weights for the b-subunit and hsp homologs are 16 and 18 kD, respectively. However, no proteins consistent with these molecular weights or pI's were overexpressed in OSA-treated M. smegmatis.

Overexpression of the b-subunit of ATP synthase indicates possible involvement of ATP synthase, whether direct or indirect, in the target pathway of OSA. Based on these observations, single time-point and time-course experiments were undertaken to determine ATP[M] levels in the presence of OSA as compared with DCCD a known, non-specific ATP synthase inhibitor. ATP[M] levels decreased significantly following OSA and DCCD treatment at all time-points tested. Not only was this decrease reproducible for both compounds, but occurred very rapidly in as little as five minutes post-exposure.

To determine if this effect was compound-specific or the result of a generalized stress response, additional antimycobacterial drugs and inhibitors of respiration were tested for their ability to affect ATP[M] level at corresponding time-points. First-line antimycobacterial agents included INH, RIF, STR, EMB, and cerulenin. Inhibitors of respiration included dicumarol (an alternative dehydrogenase inhibitor), Rot (a complex I inhibitor), and TTFA (a complex II inhibitor). All first-line drugs were used at comparable levels to that of OSA (16× their respective MIC's in BCG). Significantly, no appreciable decrease in ATP[M] level was detected at five-minutes post-exposure for any of the first-line drugs or respiratory inhibitors tested. The one exception was TTFA, a specific inhibitor of complex II, which demonstrated a moderate decrease in ATP[M] level at five minutes. This is not surprising as this inhibitor specifically targets succinate dehydrogenase (complex II), which is an integral part of the TCA cycle. Without this enzyme complex the TCA cycle is severely impaired. As a result, aerobic respiration becomes impaired, slowing the production of ATP.

Thus, OSA mimicked the effect of the well-documented, non-specific ATP synthase inhibitor DCCD. In comparison, other antimycobacterial agents failed to elicit a similar effect Similar studies were conducted with the following compounds:

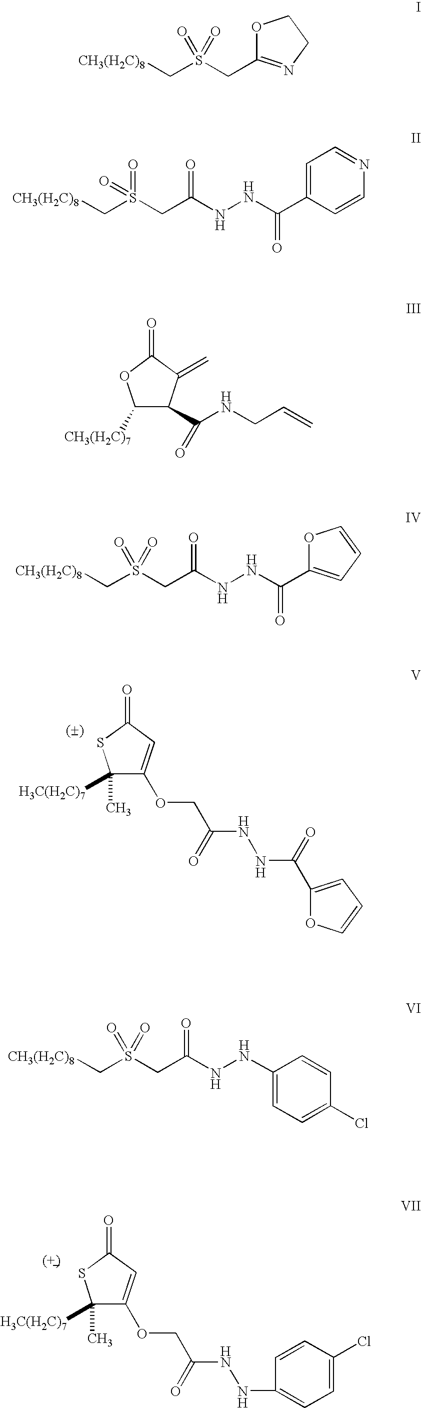

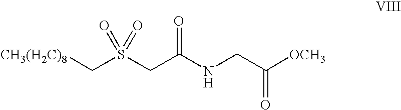

Testing of these compounds revealed similar results as was shown with OSA; that is, they showed an ability to decrease ATP[M] levels at corresponding time-points, as compared with controls.

The OSA induced decrease in ATP level was accompanied by overexpression of hsp (Rv0251c). Without limiting the scope of the invention, this suggests the possibility that the heat shock response may be linked to energy sensing/regulation in mycobacteria. Hsp (Rv0251c) encodes a relatively small protein of 159 amino acids and is a member of the Hsp20 or □-crystallin family of small heat shock proteins. Recently, Stewart et al (2002), demonstrated that hsp (termed acr2 by the authors) was the most heat-inducible gene in the mycobacterial genome. Hsp is also arranged in an apparent operon with Rv0250c and Rv0249c. Regulation of hsp involves the heat shock repressor, HspR and an ECF sigma factor σ^{E}. The latter is also upregulated during oxidative or detergent stress and bears masked similarity to the α-crystallin (acr) (14 kDa antigen) of M. tuberculosis (41% identity over 98 amino acids). The heat shock response is ubiquitous and allows cells to survive under both normal and deleterious stress conditions. This survival often requires global changes in gene expression. Most heat shock proteins are regarded as molecular chaperones, which assist in protein folding/degradation and prevent protein aggregation. In general, heat shock proteins have relatively large substrate specificity. However, emerging evidence has identified the existence of enzyme-specific chaperones, which are essential for the formation of specific enzyme complexes. Examples of enzyme-specific chaperones include the yeast ATP10, ATP11, and ATP12 genes, which encode proteins required for ATP synthase assembly. Additional enzyme-specific chaperones have been identified which are necessary for the formation of cytochrome oxidase, succinate reductase (complex II), and NADH-ubiquinone oxidoreductase (complex I). Many of these enzyme-specific chaperones fall into the Hsp20 class of molecular chaperones. Additionally, some molecular chaperones are subject to redox-regulation. The complete functional role of the mycobacterial hsp is largely unknown. However, the possibility exists that this heat shock protein may play a role in enzyme-specific assembly/regulation of ATP synthase or other associated complexes in the respiratory chain. Hsp may also represent a mycobacterial version of a redox-regulated heat shock protein.

The conclusion of OSA-mediated interference in central energy metabolism was further strengthened by the potentiation of activity with ethanol. It is known that mycobacteria can utilize low concentrations of ethanol and other short chain alcohols as carbon sources. Ethanol is a respiratory substrate, which is reversibly oxidized to acetaldehyde with the concomitant reduction of NAD by alcohol dehydrogenase. Subsequent oxidation of acetaldehyde yields acetic acid, which is then converted to acetyl-CoA in an ATP dependent reaction. Acetyl-CoA is a critical molecule in central metabolism. Oxidation of acetyl-CoA via the TCA cycle drives the production of cellular energy. Thus, ethanol metabolism and respiration are interconnected. Previous investigators have shown that ethanol increases the rate of ATP synthesis in mammalian mitochondria as a result of increased production of NADH+H⁺ which leads to elevated proton flux through the respiratory chain. ATP/O ratios increase following addition of ethanol, which indicates an increased energetic conversion between respiration and ATP synthesis. In this study, it is unlikely that ethanol and OSA share the same target. However, ethanol elevated acetyl-CoA and NADH+H⁺ would be deleterious to the cell in the event that ATP synthase or other components of the respiratory chain were impaired. In such a case, potentiation of OSA and ethanol would be possible.

Inhibition of ATP synthesis and interference with cellular respiration could produce multiple downstream effects. These include a decrease in the energy-dependent synthesis of other macromolecules, such as mycolic acids. Previously, we reported that OSA decreased mycolic acid levels in BCG, with no apparent effect on intermediates in this biosynthetic pathway. This observation was in stark contrast to the pattern of mycolate inhibition observed with thiolactomycin and cerulenin, known fatty acid synthase inhibitors. These findings indicate that inhibition of mycolic acid synthesis by OSA and other □-sulfonylcarboxamides could involve an alternative mechanism other than fatty acid synthase inhibition.

Use of compounds which can selectively decrease ATP levels, like OSA and compounds I-VIII, will aid in treating both patients presently suffering from TB (including MDR-TB), and the millions of potential patients who harbor quiescent disease which may become active as a result of immunosuppression or other systemic disease.

Such compounds may also be used against a variety of other microorganisms, such as M. avium-intracellulare, M. leprae, M. paratuberculosis, M. ulcerans, and Rhodococcus, and may be used in both humans and animals, such as horse, cattle, sheep, goats, and other ruminants.

Treatment according to the invention involves administering a compound which selectively decreases ATP levels in microorganism to a treatment subject. Pharmaceutical compositions containing any such compounds may be administered by parenteral (subcutaneously, intramuscularly, intravenously, intraperitoneally, intrapleurally, intravesicularly, or intrathecally), topical, oral, rectal, nasal or inhalation route, as necessitated by the compound, pharmaceutical carrier, or disease.

The compounds are preferably formulated in pharmaceutical compositions containing the compound and a pharmaceutically acceptable carrier. The concentration of the active agent will depend on its solubility in the carrier, and may be readily determined by a person of ordinary skill in the art. Similarly, the dose used in a particular formulation will be determined by the particular microbe against which it will be employed. The pharmaceutical composition may comprise other components, so long as they do not negate the effectiveness of the active compound. Pharmaceutically carriers are well known, and a person of skill in the art can select the correct one(s) depending on the particular route of administration.

Dose and duration of therapy will depend on a variety of factors, including the therapeutic index of the drugs, disease type, patient age, patient weight, and tolerance of toxicity. The dose will usually be chosen to achieve serum concentration levels from about 1 ng to 100 μg/ml typically 0.1 μg/ml to 10 μg/ml. Preferably, initial dose levels will be selected based on their ability to achieve ambient concentrations shown to be effective in in vitro and in vivo models and in clinical trials. The dose of a particular drug and duration of therapy for a particular subject can be determined by a skilled clinician using standard pharmacological approaches in view of the above factors. The response to the treatment may be monitored by analysis of blood or body fluid levels of the active compound, measurement of activity of the compound or its levels in relevant tissues, or monitoring the disease state of the subject. The skilled clinician will adjust the dose and duration of therapy based on the response to treatment revealed by these measurements.

The compound will, of course, be administered at a level below the level that would kill the subject, and preferably at a level below that which would irreversibly injure vital functions. Administration at a level that kills some of the patient's cells which can be regenerated (e.g., endometrial cells) is not excluded.

### EXAMPLES

The following examples are provided to illustrate, but not limit, the scope of the invention.

Mycobacteria and growth conditions. Mycobacterium tuberculosis (H37Rv) M. bovis BCG (BCG, Pasteur strain, ATCC 35734) and M. smegmatis (mc² 6 1-2c) were used in this study. Strains were maintained on Lowenstein-Jensen agar slants or Middlebrook 7H10 agar plates (Difco, Detroit, Mich.). For all assays, BCG cultures were grown at 37° C. on a rotary shaker to mid-log phase (OD=A₆₀₀ 0.3-0.4).

Compounds. Stock and working solutions of n-Octanesulfonylacetamide (OSA, Craig Townsend, Johns Hopkins University; Baltimore, Md.), dicyclohexylcarbodiimide, an ATP synthase-specific inhibitor (DCCD, ICN, Costa Mesa, Calif.), thenoyltrifluoroaceton, a respiratory complex II inhibitor (TTFA, ICN), rotenone, a respiratory complex I inhibitor (Rot, ICN), and cerulenin, a fatty acid synthase inhibitor (Sigma-Aldrich, St Louis, Mo.), were made up in dimethylsulfoxide (DMSO, Sigma). Stock solutions of isoniazid (INH), streptomycin (STR), and ethambutol (EMB) (all from Sigma) were prepared in sterile water. Initial stock solutions of rifampin (Sigma) were made up in methanol with subsequent dilutions in sterile water.

Preparation of Compounds I, II, IV, VI, and VIII

The synthesis of each of these compounds started with 3-sulfonylundecanoic acid (“IX”), which was prepared following the procedure described in J. Med. Chem. 2000 43(17) 3304.

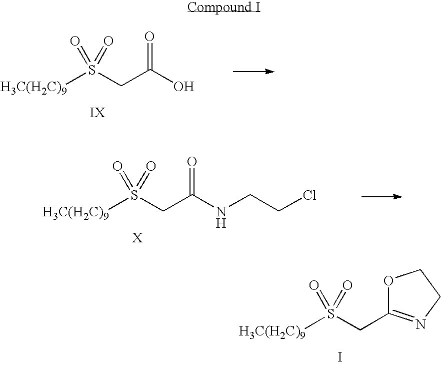

To a flame dried round bottom flask containing 3 mL of dry methylene chloride was added 3-sulfonylundecanoic acid IX, 150 mg, 0.6 mmol, and 1,1 carbonyldiimidazole (CDI), (102 mg, 0.63 mmol, under an inert atmosphere. The mixture was stirred at room temperature for 20 minutes. 2 -chloroethylamine hydrochloride (73 mg, 0.63 mmol) was then added and the reaction stirred an additional 3 hrs. Aqueous work-up provide to crude amide X (145 mg, 88%) in satisfactory yields. Amide X was used crude for further chemistry. ¹H (CDCl₃, 300 MHz) δ 6.86 (bs, 1H), 3.87 (s, 2H), 3.67-3.65 (m, 4H), 3.15 (t, J =8.1 Hz, 2H), 1.89-1.80 (m, 2H), 1.40-1.26(m, 14H), 0.88 (t, J =6.7 Hz, 3H). Amide X (110 mg, 0.33 mmol) was dissolved in 1.5 mL of methanolic potassium hydroxide (5% w/v). After stirring at ambient temperature for 2 hrs, the mixture was diluted with water and extracted three times with ethyl acetate. The organic layer was washed twice with brine, dried and-concentrated in vacuo. The crude oxazoline was purified by flash column chromotography (1:1 Hex/EtOAc) to provide 70 mg, 73% yield, of the desired product I; mp 53-55° C.; ¹H (CDCl₃, 400MHz) δ 4.37 (t, J=9.6 Hz, 2H), 3.95 (s, 2H), 3.95 (t, J=9.4 Hz, 2), 3.22 (t, J=8.2 Hz, 2H), 1.90-1.79 (m, 2H), 1.46-1.42 (m, 2H), 1.30-1.23(m, 12H), 0.87 (t, J=6.0 Hz, 3H).

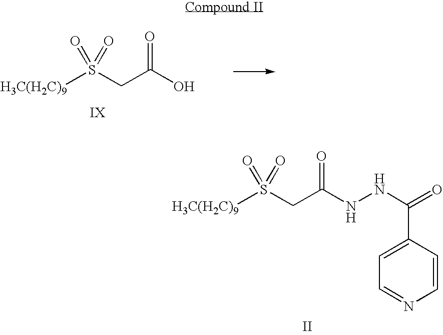

To a flame dried round bottom flask containing 3 mL of dry methylene chloride was added 3-sulfonylundecanoic acid IX (150 mg, 0.6 mmol) and CDI (102 mg, 0.63 mmol) under an inert atmosphere. The mixture was stirred at room temperature for 20 minutes. Isonicotinic hydrazide (86 mg, 0.63 mmol), was then added and the reaction stirred an additional 6.5 hrs. The reaction mixture was concentrated in vacuo and crude hydrazide II purified by flash chromatography (98% EtOAc/2% acetic acid) to give a white solid, 122 mg, 56%. ¹H (DMSO-d₆, 400 MHz) δ 10.98 (bs, 1H), 10.57 (bs, 1H), 8.77 (bs, 2H), 7.78 (d×d, J₁=1.2 Hz, J₂=5.4 Hz, 2H), 4.21 (s, 2H, 3.30 (t, J=8 Hz), 1.76-1.68 (m, 2H), 1.42-1.34 (m, 2H), 1.30-1.23 (m, 12 H), 0.84 (t, J=6.8 Hz)

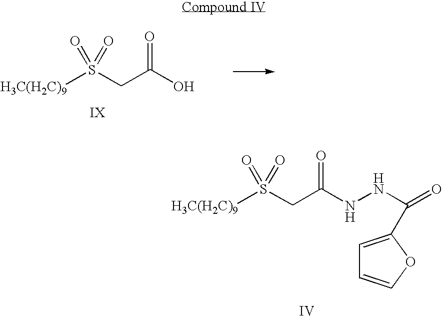

To a flame dried round bottom flask containing 3 mL of dry methylene chloride was added 3-sulfonylundecanoic acid, 158.4 mg (0.6 mmol, and 1,1 carbonyldiimidazole (CDI), 115.7 mg (0.72 mmol), under an inert atmosphere. The mixture was stirred at room temperature for 20 minutes. 2-Furoic hydrazide, 90 mg (0.72 mmol, was then added and the reaction stirred an additional 3 hours. The reaction mixture was diluted with ethyl acetate and washed twice with saturated sodium bicarbonate, three times with dilute HCl, and once with saturated NaCl. The organics were dried with magnesium sulfate and concentrated under reduced pressure. The crude product IV was recrystallized from EtOAc/Hex (3:1) to give a light brown powder (147 mg, 66%); mp 130-131° C.; ¹H (DMSO-d₆, 400 MHz) δ 10.53 (s, 1H), 10.38 (s, 1H), 7.90 (d×d, J₁=0.4 Hz, J₂=1.6 Hz, 1H), 7.24 (d×d, J1=0.6 Hz, J2=3.4 Hz), 6.65 (d×d, J1=1.6 Hz, J2=3.6 Hz), 4.17 (s, 1H), 3.28 (t, J=7.8 Hz, 2H), 1.75-1.67 (m, 2H), 1.42-1.33 (m, 2H), 1.30-1.23 (m, 12H), 0.84 (t, J=6.8 Hz, 3H).

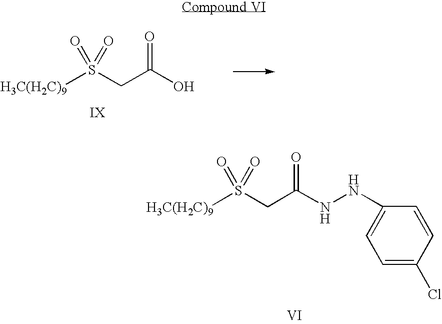

To a flame dried round bottom flask containing 3 mL of dry nethylene chloride was added 3-sulfonylundecanoic acid (163 mg, 0.62 mmol), CDI (113.9 mg, 0.74 mmol), under an inert atmosphere. The mixture was stirred at room temperature for 20 minutes. Triethylamine (TEA) (89 μL, 0.63 mmol), and 4-chlorophenylhydrazine hydrochloride (115 mg, 0.62 mmol) were then added and the reaction stirred an additional 2 hours. The crude product VI was purified by flash chromatography (40% EtOAc/60% Hex). (112 mg, 46% yield). ¹H (DMSO-d₆, 400 MHz) δ 10.13 (d, J=2.4 Hz, 1.H), 8.14 (d, J=2.4 Hz, 1 H, 7.18-7.14 (m, 2H), 6.78-6.72 (m, 2H), 4.12(s, 2H), 3.24 (t, J=7.8 Hz, 3H), 1.74-1.66 Hz (m, 2H), 1.40-132 (m, 2H), 1.30-1.23 (m, 12H), 0.84 (t, J=6.8 Hz, 3 H).

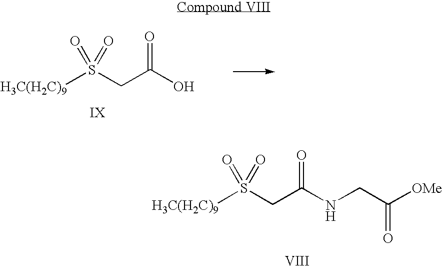

To a flame dried round bottom flask containing 3 mL of dry methylene chloride was added 3-sulfonylundecanoic acid (300 mg, 1.1 mmol), CDI (214 mg, 1.3 mmol), under an inert atmosphere. The mixture was stirred at room temperature for 20 minutes. TEA (154 μL, 1.1 mmol), and methyl glycinate hydrochloride (138 mg, 1.1 mmol) were then added and the reaction stirred an additional 4 hours. The crude ester VIII was purified by flash chromatography (50-100% EtOAc/Hex) to give a white solid (202 mg, 56%); mp 82-84° C.; ¹H (CDCl₃, 400 MHz) δ 7.13 (bs, 1H), 4.07 (d, J=6 Hz, 2H), 3.93 (s, 2H), 3.77(s, 3H), 3.23 (t, J=8 Hz, 2H), 1.90-1.82 (2H, m) 1.46-1.40 (m, 2H), 1.30-1.25(m, 12 H), 0.87 (t, J=6.8 Hz, 3 H); ¹³C (CDCl₃, 100MHz).

Preparation of Compound III

This compound was prepared in a 2 step synthesis by first preparing compound 7 from (±)-α-Methylene-γ-butyrolactone-5-octyl-4-carboxylic acid (C7.5) C75 may be prepared by as set forth in US. Pat. No. 5,981,575.

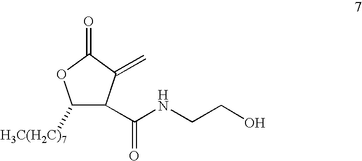

To a solution of C75, (30 mg, 0.12 mmol) in CH₃CN (0.9 mL) was added tris (2-oxo-3-oxazolinyl)phosphine oxide (91.7mg, 0.2 mmol), ethanolamine (7.8 μl, 0.13 mmol) and NEt₃ (0.04 mL, 0.3 mmol) and the solution was allowed to stir for 30 min at rt. The mixture was poured into a solution of NH₄Cl₍ₛₐₜ₎/1 N HCl (10 mL, 3:1) and extracted with Et₂O (3×15 mL). The combined organics were dried (MgSO₄), filtered, evaporated and chromatographed (35% EtOAc/Hexanes) to give compound 7 (32 mg, 91%) after flash chromatography (50% EtOAc/Hexanes-100% EtOAc/2% CH₃CO₂H). ¹H NMR (300 MHz, CDCl₃) δ 0.86 (t, J=6.9 Hz, 3 H), 1.24 (s, 10 H), 1.35-1.48 (m, 2 H), 1.64-1.75 (m, 2 H), 3.40-3.57 (m, 3H), 3.74 (t, J=5 Hz, 2 H), 4.73-4.79 (dt, J=5.7, 7 Hz, 1 H), 5.82 (d, J=2 Hz, 1 H), 6.42 (d, J=2 Hz, 1 H).

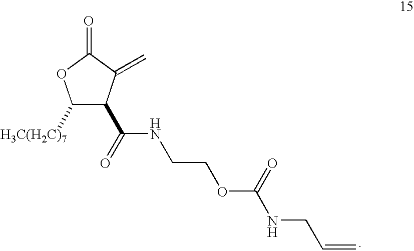

To 7 (44.9 mg, 0.15 mmol) in CH₂Cl₂ (0.7 mL) was added dimethylaminopyridine (DMAP, 4 mg, 0.03 mmol) and allyl isocyanate (20 □ L, 0.22 mmol) and the solution was allowed to stir at room temperature for 1 h. The mixture was poured into NH₄Cl (sat 10 mL) and extracted with CH₂Cl₂ (3×10 mL). The organics were combined, dried (MgSO₄) and evaporated to provide crude 15. Flash chromatography (EtOAc) provided pure 15 (19 mg, 33%). ¹H NMR (300 MHz, CDCl₃) δ 0.85 (t, J=6 Hz, 3 H), 1.24 (m, 11 H), 1.35-1.48 (m, 1 H), 1.62-1.79 (m, 2 H) 3.38-3.40 (m, 1 H), 3.51-3.52 (m, 2 H), 3.78 (t, J=5.2 Hz, 2 H) 4.20-4.21 (m, 2 H), 4.73-4.79 (m, 1 H), 4.96 (bt, 1 H), 5.09-5.20 (m, 2 H), 5.75-5.86 (m, 1 H), 5.79 (d, J=2.3 Hz, 1H), 6.38 (d, J=2.3 Hz, 1 H).

Preparation of Compounds V and VII

This compound was prepared in a synthesis with a number of intermediates. In the first step, compound 23 was prepared as follows:

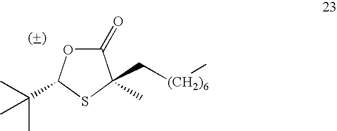

To a mixture of LiHMDS (6.2 mL 6.20 mmol, 1 M in THF) in THF (9.7 mL) at −78 ° C. was added (±)−1 (1.00 g, 5.75 mmol) in THF (9.60 mL) by cannula dropwise, and the resulting solution stirred for 30 minutes. at −78° C. Then, octyl triflate (1.63 g, 6.20 mmol) in THF (4 mL) at −78° C. was added via cannular After stirring at −78° C. for 2 hours, 1 N HCl (10 mL) was added and the solution was extracted with Et₂O (3×15 mL). The combined organics were dried (MgSO₄), filtered and evaporated. Flash chromatography (2% EtOAc/Hexanes) gave pure 23 as a 2:1-6:1 mixture of separable diastereomers (1.33 g, 81%). ¹H NMR (300 MHz, CDCl₃) δ 0.86 (t, J=6.5 Hz, 3 H), 0.99 (s, 9 H), 1.24-1.26 (m, 12 H), 1.54 (s, 3H), 1.72-1.84 (m, 2 H), 5.13 (s, 1 H); ¹³C NMR (75 MHz, CDCl₃) δ 13.9, 22.6, 24.9, 25.1, 25.9, 29.2, 29.3, 29.5, 31.8, 35.2, 41.2, 55.3, 86.5, 177.7. IR (NaCl) 3443, 2929, 1829, 1769 cm⁻¹; Analysis Calculated for C₁₆H₃₀O₂S: C, 67.0; H, 10.6. Found C, 66.3; H, 10.5. HRMS (EI) m/z calculated for C₁₆H₃₀O₂S⁺ (M+) 286.1967 obsd. 286.1969.

Then, compound 26 was prepared:

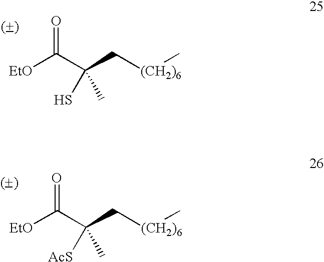

To 23 (650 mg, 2.27 mmol) in EtOH (14.1 mL) was added NaOEt (2.1 M) (2.16 mL, 4.54 mmol) (freshly prepared from Na metal (200 mg, 8.3 mmol) in EtOH (4.0 mL)) and the solution was allowed to stir at room temperature. After 2 hours, the solution was poured into NH₄Cl₍ₛₐₜ₎/1N HCl (25 mL, 3:1) and this mixture was extracted with Et₂O (3×20 mL). The combined organics were then washed with H₂O (3×25 mL), dried (MgSO₄), filtered and evaporated to give crude 25. To 25 dissolved in CH₂Cl₂ (26 mL) at 0° C. was added NEt₃ (0.5 mL, 3.49 mmol) and alkynyl chloride (0.3 mL, 3.49 mmol). After 40 minutes at 0° C., NH₄Cl₍ₛₐₜ₎ (30 mL) was added and the solution was extracted with CH₂Cl₂. The combined organics were dried (MgSO₄), filtered and evaporated. Flash chromatography (5% EtOAc/Hexanes) gave pure 26 (542 mg, 79%). ¹H NMR (300 MHz, CDCl₃) δ 0.87 (t, J=6.9 Hz, 3 H),; 1.22-1.27 (m, 15 H), 1.61 (s, 3 H), 1.75-1.84 (m, 2 H), 2.26 (s, 3 H), 4.18 (q, J=7.1 Hz, 2 H); ¹³C NMR (75 MHz, CDCl₃) δ 13.9, 14.1, 22.6, 23.4, 24.4, 29.1, 29.2, 29.6, 30.3, 31.8, 38.3, 55.8, 61.5, 173.1, 195.8. IR (NaCl) 3430, 1868, 1693, 1644 cm₋₁; Analysis Calculated. for C₁₅H₂₈O₃S: C, 62.5; H, 9.78. Found: C, 62.6; H, 9.83.

From compound 26, compound 32 was prepared.

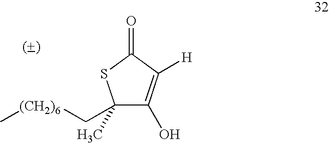

To 26 (500 mg,1.7 mmol) in toluene (27 mL) at −78° C. was added LiHMDS (4.3 mL, 4.3 mmol), 1.0 M in THF) and the solution was allowed to slowly warm to −5° C. The solution was then poured into 1 N HCl (40 mL) and extracted with EtOAc (3×25 mL). The combined organics were dried (MgSO₄), filtered and evaporated. Flash chromatography (20% EtOAc/2% CH₃CO₂H/Hexanes) gave 32 (308 mg, 73%). ¹H NMR (300 MHz, CDCl₃) keto-tautomer) δ 0.86 (t, J=6 Hz, 3 H), 1.19-1.24 (m, 10 H), 1.48-1.53 (m, 2 H), 1.65 (s, 3 H), 1.77-1.85 (m, 1 H), 1.94-2.01 (m, 1 H), 3.36 (s, 2 H); 1H NMR (300 MHz, MeOD) (enol tautomer) 0.87-0.89 (m, 3 H), 1.29 (m, 10 H), 3.29 (s, 3 H), 1.81-1.87 (m, 2 H); ¹³C NMR (75 MHz, MeOD) (enol tautomer) δ 14.7, 23.8, 26.4, 27.1, 30.5, 30.6, 30.8, 33.2, 39.8, 61.3, 103.1 (m), 189.8, 197.8. IR (NaCl) 3422, 1593 cm⁻¹; Analysis Calculated for C₁₃H₂₂O₂S: C, 64.4; H, 9.15. Found: C, 64.3; H. 9.10.

Then, compound 50 was prepared:

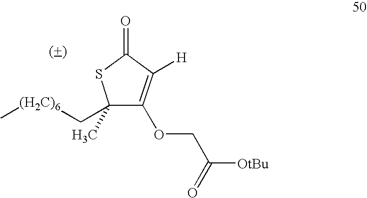

From 32 (60 mg, 0.25 mmol) and tert-butyl bromoacetate (73 □L, 0.49 mmol)d following general procedure H, was obtained 50 (62 mg, 70%) after flash chromatography (15% EtOAc/Hexanes). ¹H NMR (300 MHz, CDCl₃) δ 0.86 (t, J=7 Hz, 3 H), 1.24 (s, 12 H), 1.49 (s, 9 H), 1.68 (s, 3 H), 1.83-1.86 (m, 2 H), 4.43 (s, 2 H), 5.19 (s, 1 H); ¹³C NMR (75 MHz, CDCl₃) □ 14.0, 22.6, 25.2 26.3, 28.1, 29.2, 29.3, 29.5, 31.8, 38.9, 59.7, 68.5, 83.4, 102.1, 165.2, 185.5, 193.4. Analysis Calculated for C₁₉H₃₂O₄S: C, 64.0; H, 9.05. Found: C, 64.1; H, 9.08.

Next, compound 53 is prepared as follows:

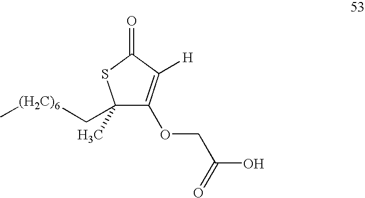

To 50 (65 mg, 0.18 mmol) dissolved in CH₂Cl₂ (1.4 mL) was added trifluoroacetic acid (TFA) (0.7 mL) and the solution was stirred at room temperature for 4 hours. The solvents were evaporated and the crude material was chromatographed (20% EtOAc/2% CH₃CO₂H/Hexanes) to give pure 53 (48 mg, 89%). ¹H NMR (300 MHz, CDCl₃) δ 0.86 (t, J=6.9 Hz, 3 H), 1.24 (s, 11 H), 1.47-1.48 (1 H), 1.68 (s, 3 H), 1.84-1.88 (m, 2 H), 4.62 (s, 2 H), 5.31 (s 1 H); ¹³C NMR (75 MHz, CDCl₃) δ 14.1, 22.6, 25.1, 26.1, 29.2, 29.3, 29.5, 31.8, 38.9, 60.1, 67.7, 102.4, 169.8, 185.8, 195.4. IR (NaCl) 3442, 1645 cm₋₁; Analysis Calculated for C₁₅H₂₄O₄S: C, 59.9; H, 8.05. Found: C, 60.0, H, 8.09.

Finally, from compound 53, compound V was prepared. To a cooled solution (0° C.) of 53 (100 mg, 0.33 mmol) in CH₂Cl₂ (1.61 mL) was added 1-[3-(dimethylamino)propyl]-3-ethylcarbodiimide hydrochloride (EDC) (128 mg, 0.43 mmol), DMAP (6.0 mg, 0.05 mmol), and 2-furoic hydrazide (54 mg, 0.43 mmol). This mixture was stirred at 0° C. for 30 minutes, then was allowed to warm to room temperature and stir for 12 h. The solution was poured into NH₄Cl (10 ml, sat) and extracted with CH₂Cl₂ (3×10 ml). The combined organics were dried (Na₂SO₄), filtered and evaporated to give crude compound V. Flash chromatography (10% EtOAc/Hex) gave pure compound V (91 mg, 68%). ¹H NMR (400 MHz, CDCl₃) δ 0.84 (t, J=6.6 Hz, 3 H), 1.21 (m, 11 H), 1.43-1.47 (m, 1 H), 1.66 (s, 3 H), 1.81-1.86 (m, 2 H), 4.64 (s, 2 H), 5.42 (s, 1 H), 6.47 (dd, J=1.6, 3.6 Hz, 1 H), 7.16 (d, J=4 Hz, 1 H), 7.45 (m, 1 H), 9.32 (d, J=4 Hz, 1 H), 9.44 (d, J=4 Hz, 1 H); ¹³C NMR (100 MHz, CDCl₃) δ 14.0, 22.6, 25.3, 26.0, 29.2, 29.3, 29.5, 31.7, 38.8, 59.7, 69.1, 103.0, 112.3, 116.5, 145.1, 145.4, 156.4, 1642, 184.8, 193.9.

Compound VII was also prepared from compound 53, as follows: To 53 (100 mg, 0.33 mmol) and 4-chlorophenylhydrazine hydrochloride (76.8 mg, 0.43 mmol) following the same general procedure as was used to prepare compound V (74 mg, 53%) after flash chromatography (50% EtOAc/Hex). ¹H NMR (300 MHz, CDCl₃) δ 0.86 (t, J=6 Hz, 3 H), 1.24 (m, 11 H), 1.46-1.54 (m, 1 H), 1.71 (s, 3 H), 1.82-1.90(m, 2 H), 4.57 (s, 2 H), 5.39 (s, 1 H), 6.75 (d, J=8.8 Hz, 2 H), 7.18 (d, J=8.8 Hz, 2 H), 7.38 (s, 1 H), 8.09 (s, 1 H); ^{3C NMR (}100 MHz, CDCl₃) □ 14.1, 22.6, 25.3, 26.1, 29.2, 29.3, 29.5, 31.8, 38.8, 59.7, 69.7, 103.2, 114.7, 126.4, 145.8, 129.2, 165.9, 184.3, 193.5. IR (NaCl) 2957, 1695, 1658, 1609 cm⁻¹.

Protein assays: mycobacteria and cultures. Either DMSO (diluent), OSA. (6.25 and 100 μg/ml), cerulenin (24 μg/ml) or isoniazid (1.0 μg/ml) were added to 20 ml control and treated cultures, respectively. Following additional incubation for 24 hours under the same conditions, 2 ml aliquots of cells were harvested by centrifugation (13,000× g for 30 seconds), the supernatant removed, and the process repeated once using 1×PBS. The cellular contents of each tube were divided into 2 to 3 Eppendorfs (roughly 3 to 5 OD units per tube), and 250 μl of a lysis solution containing 3M urea (Sigma), 0.5% Triton X-100 (Sigma), 500 mg dithiothreitol (DTT) (Gibco BRL, Life Technologies, Gaithersburg, Md.) and 500 μl Pharmalyte (Pharmacia Biotech, Piscataway, N.J.) was added. Subsequently, phenylmethylsulfonyl fluoride (PMSF) (Sigma) (100 μg/ml) and leupeptin (2 μg/ml) (Sigma) were also added. Mixtures containing cells and lysis solution were beadbeaten at maximum speed for 60 seconds in a Biospec Mini-8 beadbeater using 200-300 μm glass beads (Sigma). This process was repeated twice per sample with 30-second intervals on ice between agitations. The contents of each tube was centrifuged (13,000× g) a minimum of 30 seconds and the protein-containing supernatant removed. Protein concentration was determined using a standardized colorimetric assay (Coomassie Plus, Pierce, Rockford, Ill.) and BSA standards (Pierce) in a Shimadzu UC-1201 spectrophotometer. Quantitated samples were aliquoted and frozen at −70° C.

Protein assays: time-course. Stock BCG (500 ml) was split into 150 ml aliquots and either DMSO or OSA (100 μg/ml) were added to respective control and treated cultures followed by incubation and aeration at 37° C. for 1 hour. Aliquots (20 ml) were removed from each culture at hourly intervals during the first 4 hours post addition of compound, with extra time-points at 16, 24 and 48 hours. Cells were harvested by low speed centrifugation, washed once in sterile distilled water, and frozen at −70° C.

Protein assays: 2-D gels and sequencing of potential targets. Approximately 250 μg of each protein sample was mixed with a solution containing 8M urea, 0.5% Triton X-100, Pharmalyte 3-10, DTT, and a few grains of Bromophenol blue (Sigma) (240 μl total volume/sample). This mixture was used to rehydrate commercially prepared acrylamide pH strips (gradient 4-7) overnight at room temperature using manufacturer's standard protocols (Pharmacia Biotech). Completely rehydrated strips were subsequently subjected to a 2-dimensional protein gel system according to standardized protocols (Pharmacia Biotech) [first dimension—16 hours at 20° C., followed by equilibration of individual gel strips in a solution containing Tris-HCl (pH 6.8), urea (8M), glycerol (30%) (Sigma), SDS (1 mg/ml)) (Sigma) and either DTT or iodoacetamide (Sigma), respectively.] Following equilibration, strips were applied to commercially prepared acrylamide gels (245×110×0.5 mm, gradient 8-18%, Pharmacia) and run for 2 hours at 15° C. following manufacturer's standard protocols (Pharmacia). Molecular weight standards (size range: 14 kD to 200 kD) were purchased from Gibco BRL, Life Technologies and 10 μl loaded per gel. Visualization of proteins was done by Coomassie blue (Sigma) staining of gels overnight. Excess stain was removed with 2 to 3 washes of methanol:water:acetic acid (9:9:2, Sigma). Proteins of interest from 4 gels were pooled and the excised gel fragments were washed twice in 50% acetonitrile. Subsequent protein sequencing was done by Harvard Microchemistry (Cambridge, Mass.).

RNA extraction. Total RNA was extracted with 1 ml of Trizol reagent (Invitrogen, Carlsbad, Calif.) from 15 ml cultures of BCG treated overnight with either DMSO (diluent) or OSA. Subsequently, bacterial cells were homogenized in a mini-beadbeater for 30 seconds (twice) and chloroform was added to the bacterial lysate. Total RNA was precipitated with iso-propanol washed with ethanol and resuspended in distilled water (DNAse, RNAse free, Invitrogen). Total RNA was digested with DNAse I (Qiagen, Valencia, Calif.) and purified with RNeasy mini-kit (Qiagen). Reverse transcription to cDNA was done using 2 μg of RNA and Suer Script II, RNAse H-reverse transcriptase (Invitrogen).

PCR protocol. PCR amplification was performed in a Perkin Elmer 2400 thermal cycler. Each PCR reaction contained 2 μl of cDNA, 2.5 mM MgCl, 0.2 mM dNTP's (Invitrogen), and 2.5 units of Taq Polymerase (Invitrogen). Amplification parameters involved 30 cycles with 1 minute at 95° C., 1.5 minutes at 60° C., and 2 minutes at 72° C. Elongation was carried out at 72° C. for 10 minutes. Subsequently, the temperature was set to 4° C. Reaction products were evaluated by agarose gel electrophoresis.

Southern hybridization. PCR products were transferred onto nylon membranes (Roche Diagnostics, Indianapolis, Ind.) by Southern blotting with 20×SSC. Subsequently, individual membranes were hybridized with a gene-specific Digoxigenin 11-dUTP labeled PCR fragment at 42° C. overnight. Probe was then removed and the membrane washed in both low (2×SSC, 0.1% SDS) and high (0.5% SSC, 0.1% SDS) stringency buffer at room temperature and 68° C. for 15 minutes (twice), respectively. The Dig labeled nucleic acid was detected using a commercially available chemiluminescent kit (Roche).

ATP assays. Either diluent or OSA were added (100 μg/ml or 16× the calculated MIC) to 120 ml BCG cultures. Additional antimycobacterial agents, were also tested at comparable concentrations to that used for OSA (16× their respective MIC's). These included each of compounds I-VIII, isoniazid (INH, 1.6 μg/ml, rifampin (RIF, 32 μg/ml, streptomycin (STR 32 μg/ml, ethambutol (EMB, 32 μg/ml), and cerulenin at two concentrations (1.5 μg/ml and 24 μg/ml). Known respiratory chain inhibitors tested included DCCD (100 μg/ml), an ATP synthase-specific inhibitor, TTFA (100 μg/ml) a respiratory complex II-specific inhibitor, Rot (25 μg/ml) a respiratory complex I-specific inhibitor, and dicumarol (DC, 7 μg/ml an alternative dehydrogenase inhibitor.

Initial single and multiple time-point assays were carried out by removing culture aliquots (30 mls) at 1, 3, and 24 hours, and placing immediately on ice. All subsequent manipulations were conducted at 4° C. Additional time courses were done at 5, 30, and 180 minutes using the same procedure. Cells were harvested by centrifugation and disrupted by bead-beating with 200-300 μm glass beads in an ATP extraction buffer (100 mM Tris, 4mM EDTA, pH 7.5) at maximum force for a total of 2 minutes. Cellular debris was removed by centrifugation (13,000 × g for 15 minutes), and the ATP containing supernatant transferred to a clean tube. A commercially available ATP bioluminescence assay (Roche Diagnostics) was used for determination of ATP level in treated versus control samples. Relative light units were measured on a Wallac Victor² ™ luminometer. Colony counts (CFU's/ml were determined for each culture by plating aliquots to M7H10-ADC agar, and incubating for 3week in 5% CO₂ at 37° C. ATP level [M] was calculated per CFU of treated versus untreated groups; relative [M] ATP were then calculated by normalizing treated values to controls. Statistical significance was calculated using a 2-tailed students' t-test.

Activity of OSA in the presence of ethanol. In vitro activity of OSA in the presence of ethanol, a respiratory substrate, was determined using a modification of the standard BACTEC radiometric growth procedure (44). Briefly, inocula were prepared from M. tuberculosis cultures maintained on Lowenstein-Jensen agar slants (Difco, Detroit, Mich.) using glass beads and commercially available diluting fluid (Becton Dickinson, Sparks, Md.). Mycobacterial suspensions were vortexed with glass beads and allowed to settle for 30 minutes. The supernatant was adjusted to a, 1.0 McFarland standard and inoculated (0.1 ml) into each BACTEC 12B bottle. OSA was added to individual bottles to the following final concentrations: 1.5 μg/ml, 3.0 μg/ml, 6.25 μg/ml, 12.5 μg/ml, and 25.0 μg/ml. The final ethanol concentration used for combination testing was 0.05%. Combinations of streptomycin (0.05 μg/ml, 1.0 μg/ml, and 2.0 μg/ml and ethanol were also tested to determine whether synergistic effects observed for OSA were compound-specific or generalizable to an alternative antimycobacterial drug. All bottles were incubated at 37° C., and the growth index (GI) of each bottle recorded daily.

Treatment of BCG with OSA and other respiratory chain inhibitors and ¹⁴C-acetate lipid pulse-labeling. BCG (50 mls) was aerobically grown at 37° C. in M7H9-ADC-Tween (Difco, Detroit, Mich.) to early log phase (OD=A₆₀₀ 0.2). At this time 1 μCi/ml of [1,2 ¹⁴C] acetic acid (Amersham, Arlington Heights, Ill.) and either diluent (DMSO), OSA (100 μg/ml), DCCD (100 μg/ml, or TTFA (100 μg/ml, were added to respective cultures and incubated under the same conditions for 10 minutes. Cultures were immediately placed on ice and cells were harvested by centrifugation at 3,000 × g for 15 minutes at 4° C.

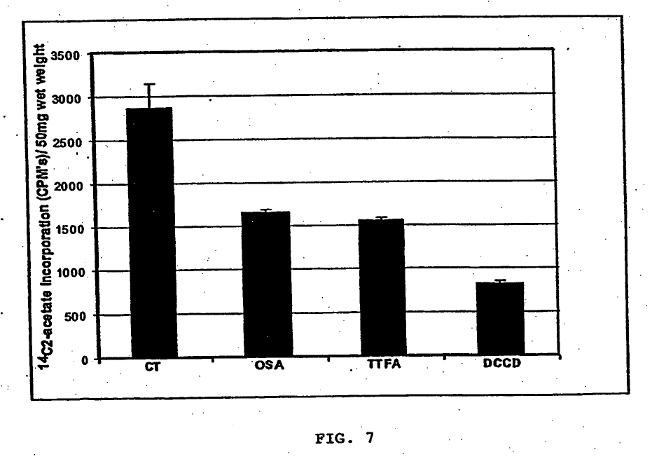

Mycolic acids preparation and analysis. Mycolic add extraction was performed as previously described in publications such as Dobson, G., et al., “Systematic analysis of complex mycobacterial lipids,” in Chemical Methods in Bacterial Systematics, p. 237-265. M. Goodfellow and D. Minnikin (eds.), Academic Press, London (1985), and Minnikin, D., et al., “Extraction of mycobacterial mycolic acids and other long-chain compounds by an alkaline methanolysis procedure,” Journal of Microbiological Methods, 2:243-249 (1984). Briefly, polar and non-polar extractable lipids were removed from equal volumes of cells (60 mg wet weight) according to established protocols from the above-references. The resulting defatted cells containing bound mycolic acids were subjected to alkaline hydrolysis in methanol (1 ml), 30% KOH (1 ml) and toluene (0.1 ml) at 75° C. overnight and subsequently cooled to room temperature. The mixture was then acidified to pH 1 with 3.60% HCl and extracted 3 times with diethyl ether. Combined extracts were dried under N₂. Fatty acid methyl esters of mycolic acids were prepared by mixing dichloromethane (1 ml), a catalyst solution (1 ml) (26), and iodomethane (25 ml), for 30 minutes, centrifuging and discarding the upper phase. The lower phase was dried under N₂. Incorporation of ¹⁴C-acetate into mycolic acids was determined by scintillation counting (Beckman LS6500 multi-purpose scintillation counter) and values expressed as a percent of untreated controls. Comparison of the effects of OSA (100 μg/ml), DCCD (100 μg/ml), and TTFA (100 μg/ml) on mycolic acid synthesis following 10 minutes exposure in early log phase cultures of BCG is shown in FIG. 7.

Results

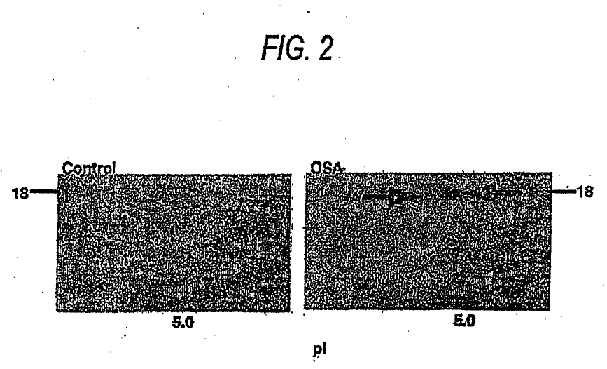

Initially, qualitative and quantitative identification of the OSA-specific protein target was attempted by examining 2-dimensional gel electrophoretic protein profiles following a 24-hour exposure to OSA in BCG. Previous investigators successfully used a similar approach to identify the enzymatic target of isoniazid in M. tuberculosis as described in Mdluli D., et al., “Inhibition of a Mycobacterium tuberculosis, β-ketoacyl ACP synthase by isoniazid,” Science, 280:1607-1610 (1998). As shown in FIG. 2 (Right), treatment with OSA resulted in significant overexpression of two relatively small proteins with approximate molecular weights of 17 to 18 kD. Both proteins were undetectable in the corresponding untreated controls (FIG. 2, Left). Overexpression occurred both at the MIC of OSA (6.25 μg/ml) and concentrations up to 16 times (16×) the MIC (100 μg/ml) in a dose-dependent manner. A separate time-course experiment using ³⁵S-methionine pulse-labeling demonstrated that both proteins were overexpressed in as little as 3.5 hours post OSA exposure. In comparison, treatment of BCG with either cerulenin or isoniazid, potent antimycobacterial inhibitors, failed to result in overexpression of either protein at concentrations up to 16× the MIC. Additionally, neither protein was overexpressed in OSA treated M. smegmatis, which is intrinsically resistant to this compound.

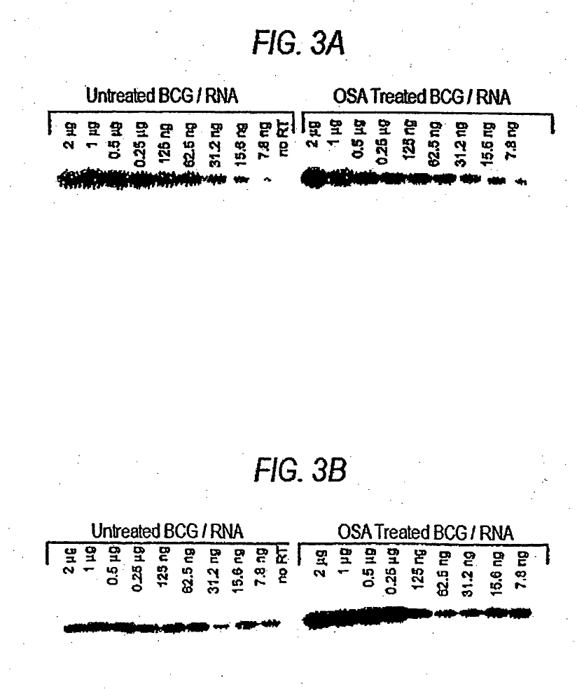

Sequencing of pooled 2-dimensional gel fragments containing each of the two proteins demonstrated the more prominent species to be a small heat shock protein (hsp, Rv 0251c) of 17,786-daltons with an isoelectric point (pI) of 5.0. The second protein was identified as the b-subunit of ATP synthase encoded by the atpF gene (Rv1306), with a molecular weight of 18,325-daltons and a pI of 4.9. Overexpression of both proteins was confirmed by RT-PCR (FIG. 3).

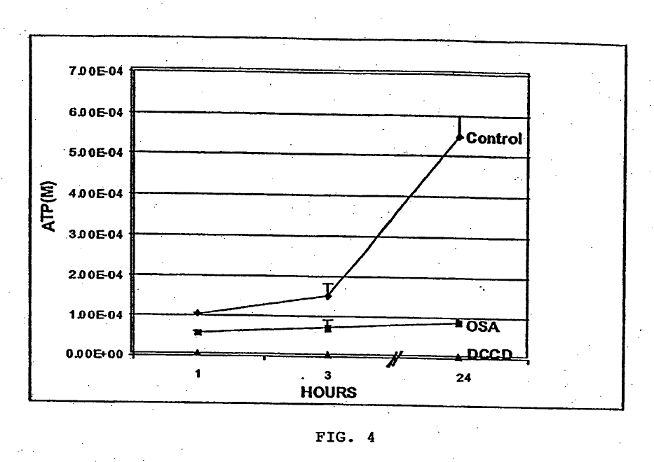

On the basis of the protein data, which suggested a possible connection to ATP synthase via interaction with the b-subunit, time-course studies over 24 hours were performed to examine the effect of OSA (100 μg/ml) on ATP levels in BCG in comparison to DCCD a known ATP synthase inhibitor. As shown in FIG. 4, ATP[M] levels decreased significantly (46%) in OSA treated BCG versus untreated controls at one hour post exposure. This trend continued at three and 24 hours, with 54% and 85% reduction in ATP [M], respectively. These differences were statistically significant at each time point (p<0.02). DCCD, an inhibitor of ATP synthase, also caused marked reduction in ATP[M] level with a 95% decrease at all timepoints (p=0.003).

Similar results were observed for each of compounds I-VIII.

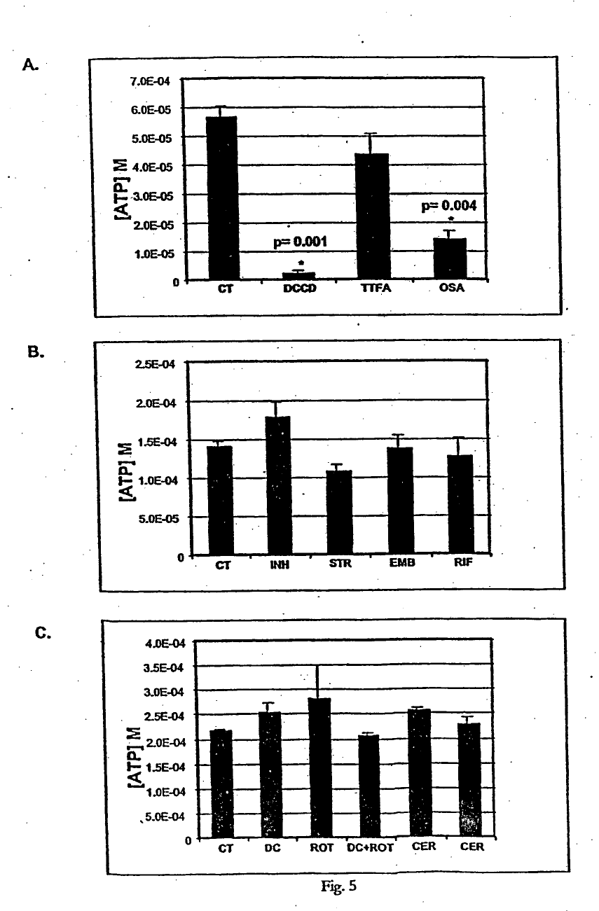

Subsequent experiments were conducted to determine how rapidly the effect of OSA on ATP[M] concentration occurred. As shown in FIG. 5 (Panel A), OSA significantly decreased the ATP level in treated versus untreated BCG in as little as 5 minutes. This difference was statistically significant (p=0.004). A statistically significant decrease (p=0.001) was also noted with DCCD, a specific ATP synthase inhibitor. Treatment with TTFA, a respiratory Complex II inhibitor, resulted in only a moderate decrease in ATP at the corresponding time-point.

To determine if the effect of OSA on ATP level could be the result of a generalized stress response in BCG, additional antimycobacterial agents such as INH, RIF, EMB, and STR, were tested at comparable concentrations to that used for OSA (16× their respective MIC's, INH 1.6 μg/ml, RIF 32 μg/ml, STR 32 μg/ml, EMB 32 μg/ml, and cerulenin 24 μg/ml). As shown in FIG. 5 (Panel B), treatment of BCG with standard antimycobacterial drugs (16× their respective MIC's) resulted in no appreciable difference in ATP level versus untreated controls at the corresponding time point of five minutes post-exposure. Additional compounds tested included dicumarol, an alternative dehydrogenase inhibitor, rotenone (Rot), specific for NADH dehydrogenase (Respiratory Complex I), and a fatty acid synthase inhibitor, cerulenin. No appreciable decrease in ATP level was noted with this group of compounds (FIG. 5, Panel C).

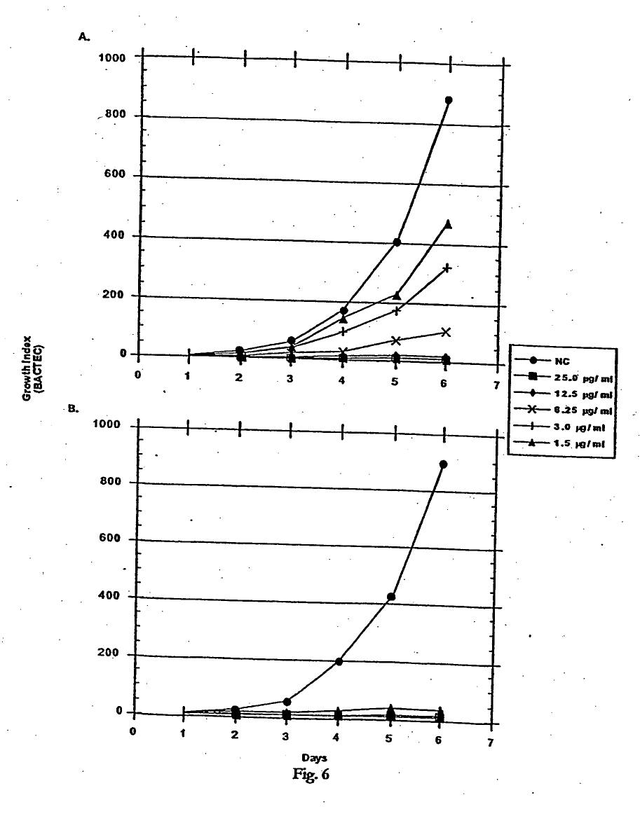

Due to the possible involvement of ATP synthase and other components of the respiratory chain, studies were formed with OSA in the presence of ethanol (0.05%). Ethanol is a respiratory substrate and has been used by multiple investigators to study cellular respiration, as shown, for example, in Beauvieux, M. P., et al., “Ethanol perfusion increases the yield of oxidative phosphorylation in isolated liver of fed rats,” Biochim. Biopbys. Acta, 570: 135-140 (2002). As shown in FIG. 6, 0.05% ethanol potentiated the effects of OSA on growth inhibition, reducing the MIC from 6.25 μg/ml in M. tuberculosis H37Rv to <1.5 μg/ml. No potentiation in activity was observed between ethanol and streptomycin. Previously we reported that treatment of BCG with OSA resulted in a decrease in mycolic acids, with no apparent effect on intermediates in this pathway. A significant decrease in ATP level could potentially have direct or indirect deleterious effects on the biosynthesis of other macromolecules, including mycolic acids of the cell wall. To investigate the role of ATP synthesis and respiration in mycolic acid production, inhibitors of ATP synthase (DCCD) and respiratory complex II (TTFA) were evaluated and compared to OSA and untreated controls in BCG. A short time interval of 10 minutes post exposure was selected to ensure that inhibition in mycolate synthesis was not due to cell death. As shown in FIG. 6, total mycolic acid levels decreased 79% with DCCD (100 μg/ml), 46% with TTFA (100 μg/ml), and 43% with OSA (100 μg/ml) compared to untreated controls. Panel A of FIG. 6 shows the activity of OSA in standard BACTEC radiometric media without ethanol (concentrations in μg/ml indicated in the legend), while Panel B shows activity of OSA, using the same concentrations and media supplemented with 0.5% ethanol. NC shows the results for an untreated control.

1. A method of treating a subject with a microbially-based infection, comprising the administration of an effective amount of a compound to a subject in need of treatment, the compound being able to decrease ATP levels in the microbe by at least 10% compared to controls after 24 hours in an in vitro test, and not kill mammalian cells during the same time period, the decrease in ATP levels being measured by:
(1) removing the cells from the testing location and putting them on ice;
(2) harvesting the cells at 4 degrees C. by centrifugation and disrupting it with bead-beating in an ATP extraction buffer;
(3) removing cellular debris by centrifugation at 4 degrees C., leaving an ATP-containing supernatant;
(4) measuring the amount of ATP present in the supernatant by a bioluminescence assay at 4 degrees C.;
wherein the compound is not of formula R—SOₙ-Z-CO—Y, wherein n is 1 or 2, R is a hydrocarbon group having 6-20 carbon atoms, Z is a hydrocarbon linking moiety that may contain a heteroatom, and Y is selected from —NH, —O—CH₂—C₆H₅, —CO—CO—O—CH₃, and —O—CH₃.

2. The method of claim 1, wherein the subject is a human.

3. The method of claim 1, wherein the subject is an animal.

4. The method of claim 3, wherein the subject is selected from the group consisting of horses, cattle, goats and sheep.

5. The method of claim 1, wherein the compound is selected from the group consisting of:

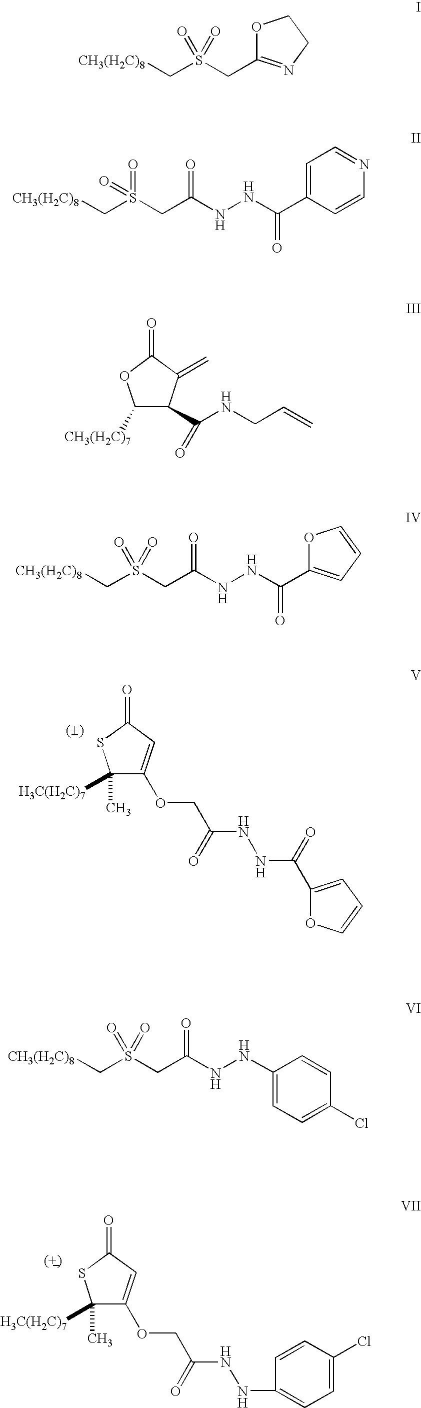

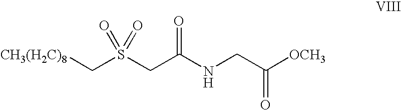

6. The method of claim 5, wherein the compound is

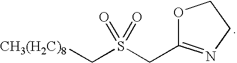

7. The method of claim 5, wherein the compound is

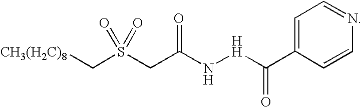

8. The method of claim 5, wherein the compound is

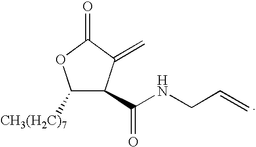

9. The method of claim 5, wherein the compound is

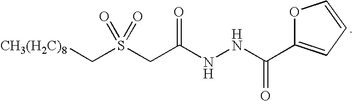

10. The method of claim 5, wherein the compound is

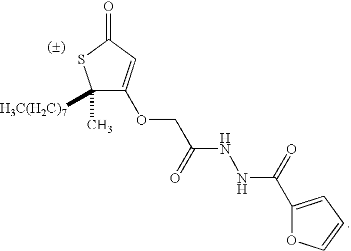

11. The method of claim 5, wherein the compound is

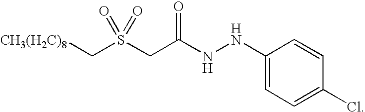

12. The method of claim 5, wherein the compound is

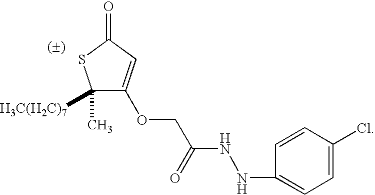

13. The method of claim 5, wherein the compound is

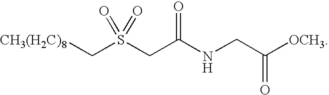

14. The method of claim 1, wherein the subject is infected with a microbe selected from the group consisting of M. tuberculosis, M. avium-intracellulare, M. leprae, M. paratuberculosis, M. ulcerans, and Rhodococcus.

15. A method of treating a subject with a microbially-based infection, comprising the administration of a compound to a subject in need of treatment, wherein the compound produces overexpression of the b-subunit of ATP synthase, and further wherein the compound is not of formula R—SOₙ-Z-CO—Y, wherein n is 1 or 2;
R is a hydrocarbon group having 6-20 carbon atoms, Z is a hydrocarbon linking moiety that may contain a heteroatom, and Y is selected from —NH, —O—CH₂—C₆H₅, —CO—CO—O—CH₃, and —O—CH₃.

16. The method of claim 15, wherein the subject is a human.

17. The method of claim 15, wherein the subject is an animal.

18. The method of claim 17, wherein the subject is selected from the group consisting of horses, cattle, and sheep.

19. The method of claim 15, wherein the compound is selected from the group consisting of:

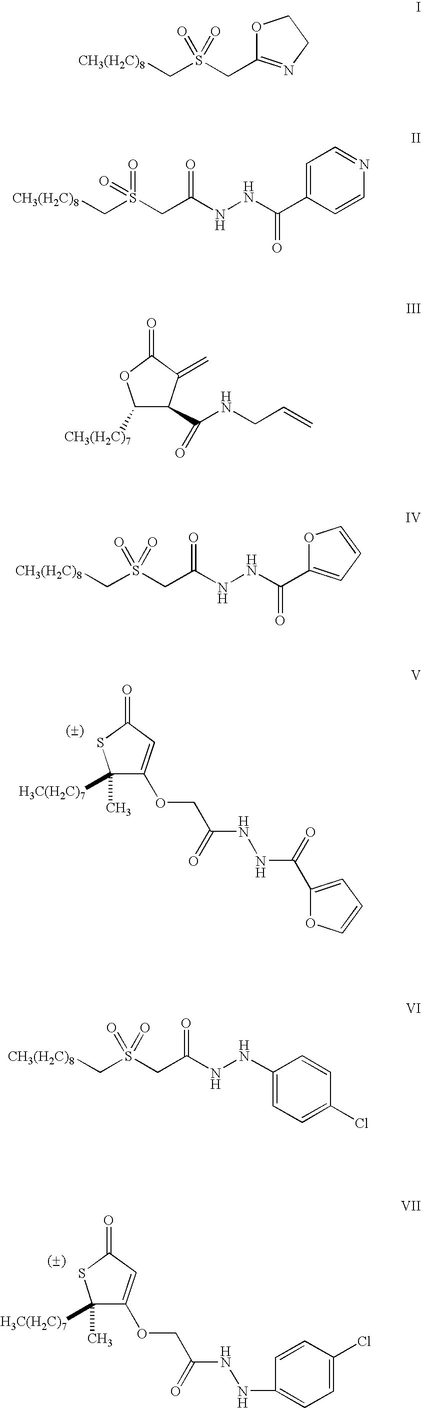

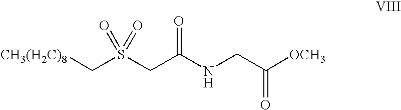

20. The method of claim 19, wherein the compound is

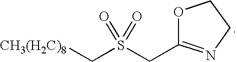

21. The method of claim 19, wherein the compound is

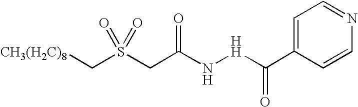

22. The method of claim 19, wherein the compound is

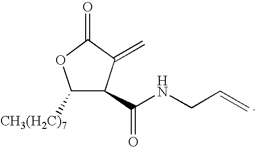

23. The method of claim 19, wherein the compound is

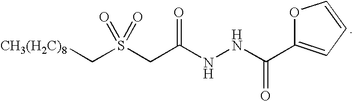

24. The method of claim 19, wherein the compound is

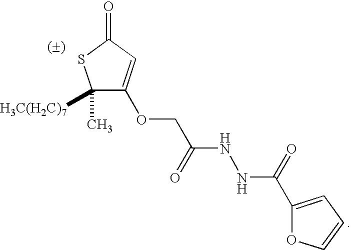

25. The method of claim 19, wherein the compound is

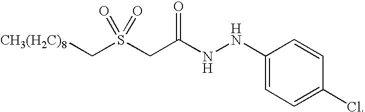

26. The method of claim 19, wherein the compound is

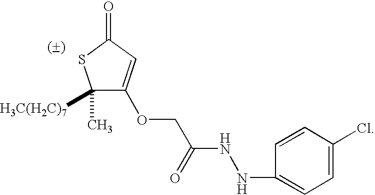

27. The method of claim 19, wherein the compound is [chemical structure]
